(19) United States
(12) Patent Application Publication
(10) Pub. No.: US 2024/0123247 A1
(43) Pub. Date: Apr. 18, 2024

(54) TREATMENT APPARATUS USING HIGH FREQUENCY HANDPIECE, AND CONTROL METHOD THEREFOR

(71) Applicant: S&M Medical Inc., Gwangju (KR)

(72) Inventors: Sham Shik SHIN, Gwangju (KR); Dae Hyun SHIN, Gwangju (KR); Hyun Jin GONG, Gwangju (KR)

(21) Appl. No.: 18/275,834

(22) PCT Filed: Feb. 5, 2021
(86) PCT No.: PCT/KR21/01584

§ 371 Date: Aug. 4, 2023

(30) Foreign Application Priority Data
Feb. 4, 2021 (KR) 10-2021-0016085

(51) Int. Cl. A61N 1/40; A61N 1/02; A61N 1/06; A61N 1/32; A61N 1/36; A61N 7/00

(52) U.S. Cl. CPC A61N 1/403; A61N 1/025; A61N 1/06; A61N 1/328; A61N 1/36031; A61N 1/36034; A61N 7/00; A61N 2007/0034

(57) ABSTRACT

A treatment apparatus using a radiofrequency handpiece and a control method therefor, and particularly, to a treatment apparatus that automatically increases the temperature of the skin surface by periodically controlling and changing the temperature of a plurality of applicators, and a control method therefor. There is an advantage of automatically increasing the temperature of the skin surface by periodically controlling and changing the temperature of a plurality of applicators.

### TECHNICAL FIELD

The present invention relates to a treatment apparatus using a radiofrequency handpiece and a control method therefor, and particularly, to a treatment apparatus that automatically increases the temperature of the skin surface by periodically controlling and changing the temperature of a plurality of applicators, and a control method therefor.

### BACKGROUND ART

Recently, technology for treating skin by modifying the state of skin tissue or improving skin tissue characteristics through the transfer of energy to the skin has been widely applied. Skin treatment devices using various energy sources such as lasers, flash lamps, and ultrasonic waves have been developed, and recently, skin treatment devices using radio-frequency (RF) wave energy have also been actively researched and developed.

Methods of treating tissue may be classified into a method of treating tissue from the outside of the tissue, and an invasive treatment method which is performed by inserting a part or all of a treatment device into tissue. Thereamong, the invasive treatment method mainly uses a treatment device having a small-diameter insertion part such as a needle or catheter, and includes various treatment actions such as delivering a therapeutic agent into tissue, or performing surgical treatment by mechanical operation in a state adjacent to a specific site inside tissue, or delivering energy to a target position inside tissue.

When RF energy is provided to skin tissue, the RF current flows along the skin tissue. The skin tissue serves as resistance, and for this reason, deep heat is generated in the skin tissue. This deep heat may raise the temperature of the skin tissue and remodel the collagen layer, thus reducing wrinkles and enhancing skin elasticity. In addition, the deep heat has an effect of improving the overall skin condition, such as preventing skin aging by enhancing the blood circulation in skin tissue.

As a conventional art in this regard, US Patent Publication No. US2005/0171581 provides a system and method that use a light source to treat tissue with NIR light. In this case, raising the temperature of the skin layer may act to induce collagen contraction or remodeling, and a light source may be used to provide a broad spectrum of NIR light, and it is possible to raise the temperature of deeper tissue layers than that of tissue layers closer to the skin surface.

However, such a conventional light source treatment or RF treatment device has problems in that, when high energy is applied within a short time, excessive energy may be applied, causing tissue damage, and the treatment effect may vary depending on the skill of the operator.

### DISCLOSURE

### Technical Problem

An object of the present invention is to provide a treatment apparatus that uses a radiofrequency handpiece so that excessive energy is not applied within a short time.

Another object of the present invention is to provide a control method of controlling the treatment apparatus by controlling the treatment time and output of each applicator provided in the handpiece.

### Technical Solution

To achieve the above objects, the present invention provides a treatment apparatus using a radiofrequency handpiece, the apparatus including: an input unit configured to receive information on a user who is to be treated with a radiofrequency; a handpiece configured to generate the radiofrequency depending on the user information, detect an ambient temperature, and increase the temperature stepwise while controlling the radiofrequency depending on a preset time, the handpiece including a plurality of applicators having different radiofrequency output modes depending on a body part of the user; and a control unit configured to control the output of the radiofrequency, which is generated by the handpiece, depending on the temperature, and to control the operating mode of the handpiece through the impedance value of a treatment area, which is fed back from the user.

Preferably, the user information may include at least one of the user's age, gender, height, weight, treatment area, or body mass index (BMI), and depending on the user information, the frequency of the radiofrequency may be initialized to operate stepwise during a preset treatment time.

According to an embodiment, the plurality of applicators may have at least one of a radiation pad, an electrode pad, a carbon sheet, or an air pad so as to operate in a monopolar radiofrequency manner and may be attached in varying numbers depending on a body part of the user, and the handpiece may further include: a frequency unit configured to output a frequency set for each section of the treatment time; and a temperature sensor unit configured to measure the temperature of the body part that increases stepwise depending on the output of the frequency unit.

According to an embodiment, the handpiece may further include a single ultrasonic vibration unit capable of generating ultrasonic waves and vibrations, and the ultrasonic vibration unit may adjust the number of repetitions for contraction and relaxation of muscles or may be operated alternately with the frequency unit.

According to an embodiment, the radiation pad and the electrode pad in the applicator may further include a urethane sheet that comes into contact with the skin, the radiation pad may have a positively charged carbon sheet attached to a lower surface of the urethane sheet, and a negatively charged carbon sheet attached to a lower surface of the electrode pad.

According to an embodiment, the temperature sensor unit may stop radiofrequency output from the corresponding applicator when a critical temperature initially set in the control unit is detected.

According to an embodiment, the control unit may determine at least one applicator set to be used for the body part, and set the operating frequency and duration of each applicator as a cycle depending on the operating time.

According to an embodiment, the control unit may set the number of repetitions of the cycle according to the duration, and control the applicators to increase the temperature of the body part stepwise while alternately operating each applicator used in the cycle.

The present invention also provides a control method for a treatment apparatus using a radiofrequency handpiece, the method including steps: allowing a control unit to receive, through an input unit, information on a user who is to be treated with a radiofrequency, and to initialize the treatment time and radiofrequency output of each applicator, provided in the handpiece, depending on the user information; generating the radiofrequency during the treatment time, detecting an ambient temperature, and increasing the temperature stepwise while controlling the radiofrequency depending on a preset time; and controlling the operation of the applicators included in the handpiece by receiving feedback from the user on the temperature depending on the output of the radiofrequency.

According to an embodiment, the step of initializing the treatment time and radiofrequency output may include steps of: determining the number of the applicators using at least one of the user's age, gender, height, weight, treatment area, or body mass index (BMI) included in the user information; determining the operation sequence of each applicator depending on the number of the applicators; and setting the operating frequency and duration of each applicator as one cycle depending on the operation sequence.

According to an embodiment, the method may include steps of: allowing the control unit to set the number of repetitions of the cycle depending on the duration; and alternately operating each applicator used in the cycle, and the temperature of the treatment area may be increased stepwise according to the operation of the applicators.

The present invention also provides a computer-readable recording medium storing a program for implementing the above-described method as a program.

### Advantageous Effects

According to the present invention having the configuration described above, there is an advantage of automatically increasing the temperature of the skin surface by periodically controlling and changing the temperature of a plurality of applicators.

### BRIEF DESCRIPTION OF DRAWINGS

FIG. 1 illustrates the appearance of a treatment apparatus using a radiofrequency handpiece according to the present invention.

FIG. 2 is a block diagram of the treatment apparatus using a radiofrequency handpiece according to the present invention.

FIG. 3 shows a cycle of the operating frequency and duration of the handpiece according to an embodiment of the present invention.

FIG. 4 is a flow chart showing a control method for the treatment apparatus using a radiofrequency handpiece according to the present invention.

FIG. 5 is a flow chart for initializing the treatment time and radiofrequency output of each applicator according to an embodiment of the present invention.

FIG. 6 is a flow chart for increasing the temperature of a treatment site stepwise according to an embodiment of the present invention.

### BEST MODE

To achieve the above objects, the present invention provides a treatment apparatus using a radiofrequency handpiece, the apparatus including: an input unit configured to receive information on a user who is to be treated with a radiofrequency; a handpiece configured to generate the radiofrequency depending on the user information, detect an ambient temperature, and increase the temperature stepwise while controlling the radiofrequency depending on a preset time, the handpiece including a plurality of applicators having different radiofrequency output modes depending on a body part of the user; and a control unit configured to control the output of the radiofrequency, which is generated by the handpiece, depending on the temperature, and to control the operating mode of the handpiece through the impedance value of a treatment area, which is fed back from the user.

Preferably, the user information may include at least one of the user's age, gender, height, weight, treatment area, or body mass index (BMI), and depending on the user information, the frequency of the radiofrequency may be initialized to operate stepwise during a preset treatment time.

According to an embodiment, the plurality of applicators may have at least one of a radiation pad, an electrode pad, a carbon sheet, or an air pad so as to operate in a monopolar radiofrequency manner and may be attached in varying numbers depending on a body part of the user, and the handpiece may further include: a frequency unit configured to output a frequency set for each section of the treatment time; and a temperature sensor unit configured to measure the temperature of the body part that increases stepwise depending on the output of the frequency unit.

According to an embodiment, the handpiece may further include a single ultrasonic vibration unit capable of generating ultrasonic waves and vibrations, and the ultrasonic vibration unit may adjust the number of repetitions for contraction and relaxation of muscles or may be operated alternately with the frequency unit.

According to an embodiment, the radiation pad and the electrode pad in the applicator may further include a urethane sheet that comes into contact with the skin, the radiation pad may have a positively charged carbon sheet attached to a lower surface of the urethane sheet, and a negatively charged carbon sheet attached to a lower surface of the electrode pad.

According to an embodiment, the temperature sensor unit may stop radiofrequency output from the corresponding applicator when a critical temperature initially set in the control unit is detected.

According to an embodiment, the control unit may determine at least one applicator set to be used for the body part, and set the operating frequency and duration of each applicator as a cycle depending on the operating time.

According to an embodiment, the control unit may set the number of repetitions of the cycle according to the duration, and control the applicators to increase the temperature of the body part stepwise while alternately operating each applicator used in the cycle.

The present invention also provides a control method for a treatment apparatus using a radiofrequency handpiece, the method including steps: allowing a control unit to receive, through an input unit, information on a user who is to be treated with a radiofrequency, and to initialize the treatment time and radiofrequency output of each applicator, provided in the handpiece, depending on the user information; generating the radiofrequency during the treatment time, detecting an ambient temperature, and increasing the temperature stepwise while controlling radiofrequency depending on a preset time; and controlling the operation of the applicators included in the handpiece by receiving feedback from the user on the temperature depending on the output of the radiofrequency.

According to an embodiment, the step of initializing the treatment time and radiofrequency output may include steps of: determining the number of the applicators using at least one of the user's age, gender, height, weight, treatment area, or body mass index (BMI) included in the user information; determining the operation sequence of each applicator depending on the number of the applicators; and setting the operating frequency and duration of each applicator as one cycle depending on the operation sequence.

According to an embodiment, the method may include steps of: allowing the control unit to set the number of repetitions of the cycle depending on the duration; and alternately operating each applicator used in the cycle, and the temperature of the treatment area may be increased stepwise according to the operation of the applicators.

The present invention also provides a computer-readable recording medium storing a program for implementing the above-described method as a program.

### MODE FOR INVENTION

The terms used in the present specification will be briefly described, and the present invention will be described in detail.

The terms used in the present invention are currently widely used general terms selected in consideration of their functions in the present invention, but they may change depending on the intents of those skilled in the art, precedents, or the advents of new technology. In addition, in certain cases, there may be terms arbitrarily selected by the applicant, and in this case, their meanings are described in detail in a corresponding description part of the present invention. Accordingly, terms used in the present disclosure should be defined based on the meaning of the term and the entire contents of the present invention, rather than the simple term name.

Throughout the present specification, it is to be understood that when any part is referred to as “including” any component, it does not exclude other components, but may further include other components, unless otherwise specified. In addition, terms such as “ . . . unit” and “module” described in the specification mean a unit that processes at least one function or operation, which may be implemented in hardware or software or a combination of hardware and software. In addition, throughout the present specification, it is to be understood that when any part is referred to as being “connected” to another part, it may be connected “directly” to the other part or “intervening elements” may be present.

Hereinafter, embodiments of the present invention will be described in detail with reference to the accompanying drawings so that they can be easily carried out by those skilled in the art to which the present invention pertains. However, the present invention may be embodied in various different forms and is not limited to the embodiments described herein. In addition, in the drawings, parts irrelevant to the description are omitted in order to clearly explain the present invention, and like reference numerals denote like parts throughout the specification.

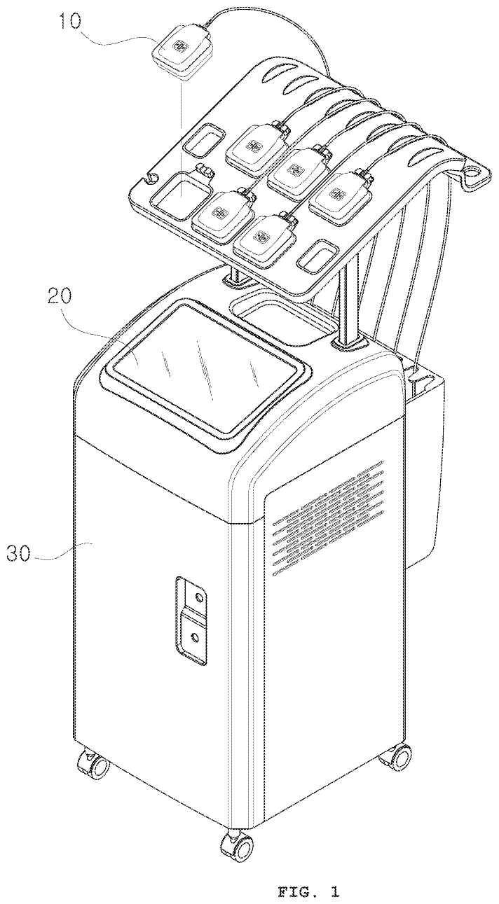

FIG. 1 shows the appearance of a treatment apparatus using a radiofrequency handpiece 10 according to the present invention.

Referring to FIG. 1, the present invention may include an input unit 20, a handpiece 10, and a control unit 30.

The input unit 20 may receive information on a user who is to be treated with a radiofrequency.

The input unit 20, a kind of control panel, may set an operating method of the control unit 30 or select various control modes that control the handpiece 10 through the control unit 30. The input unit 20 may be composed of a touch screen panel and may be formed integrally with a display. Alternatively, a separate display may be provided.

The handpiece 10 may generate the radiofrequency depending on the user information, detect an ambient temperature, and increase the temperature stepwise while controlling the radiofrequency depending on a preset time, and.

The handpiece 10 may include a plurality of applicators 11 having different radiofrequency output modes depending on a body part of the user. In an embodiment of the present invention, the handpiece 10 may include six applicators 11.

In the present invention, the handpiece 10 refers to the applicator 11, and the applicator 11 in a narrow sense refers to a device that comes into contact with the patient's treatment area, and may, in a broad sense, include, in addition to the applicator 11, various electrodes or a temperature sensor unit 13 connected to the applicator 11.

The control unit 30 may control the output of the radiofrequency, which is generated by the handpiece 10, depending on the temperature, and control the operating mode of the handpiece 10 through the impedance value of the treatment area, which is fed back from the user. Although not shown in detail in the drawing, the control unit is generally provided inside the body of the apparatus, and may generally include a control means for adjusting the operation of the handpiece 10.

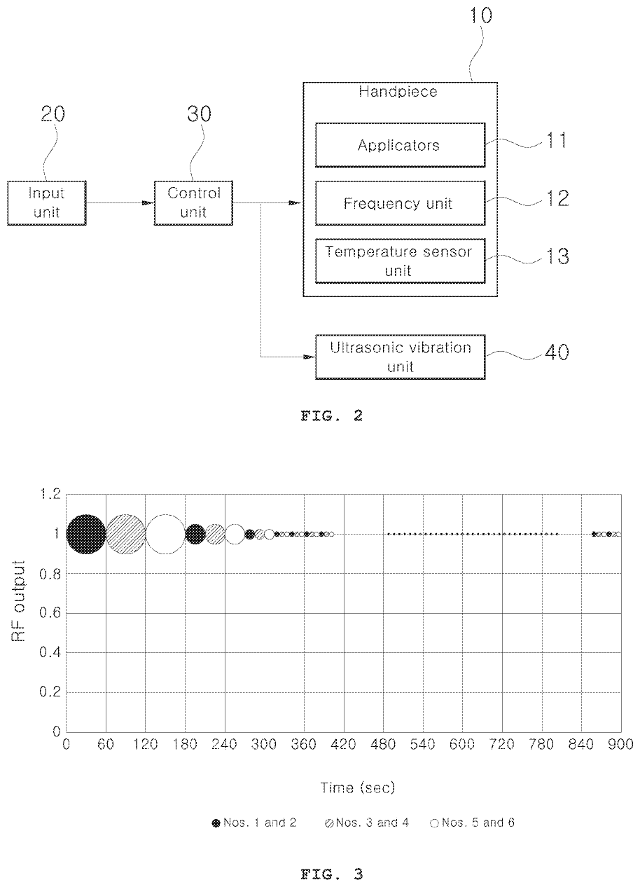

FIG. 2 shows a block diagram of the treatment apparatus using the radiofrequency handpiece 10 according to the present invention.

Referring to FIG. 2, the present invention may control the handpiece 10 through the input unit 20 and the control unit 30, and may also control an ultrasonic vibration unit 40 in addition to the handpiece 10.

The input unit 20 may be composed of a control panel or a display panel, and may receive user information.

The input unit 20 may receive user information. Here, the user is a person receiving treatment, and the user information may include at least one of age, gender, height, weight, treatment area, or body mass index (BMI).

Depending on the user information, the frequency of the radiofrequency may be initialized to operate stepwise during a preset treatment time.

According to an embodiment, when the user information changes, the sensitivity according to the transfer of temperature may change, and the mode of applying the temperature may be changed depending on the treatment area. That is, in the present invention, when user information is input, various body organs may be selected, and the operating mode of the applicator 11 may be set differently stepwise depending on each body organ and the user's condition.

The handpiece 10 may operate in a monopolar radiofrequency manner. According to an embodiment, the applicator may include at least one of a radiation pad, an electrode pad, a carbon sheet, or an air pad. These pad may be placed in contact with a user's tissue surface and may include a plurality of therapeutic electrodes configured to deliver RF energy.

In the applicator 11, the radiation pad and the electrode pad may further include a urethane sheet that comes into contact with the skin, and the radiation pad may have a positively charged carbon sheet attached to a lower surface of the urethane sheet, and a negatively charged carbon sheet attached to a lower surface of the electrode pad.

Particularly in the case of the applicator 11, the plurality of therapeutic electrodes may include at least two individually addressable therapeutic electrodes to which different therapeutic RF signals may be applied. The RF signals may represent one or more of power, duty cycle, pulse duration, phase, and RF frequency, as described in detail with reference to FIG. 3.

The handpiece 10 may include: a plurality of applicators 11 that may be attached in varying numbers depending on a body part of the user; a frequency unit 12 configured to output a frequency set for each section of the treatment time; and a temperature sensor unit 13 configured to measure the temperature of a body part that gradually rises depending on the output of the frequency unit 12.

The frequency unit 12 and the temperature sensor unit 13 may be connected to the plurality of applicators 11, and the frequency unit 12 is configured so that it may receive power from a power supply unit and generate RF energy or frequency. The frequency unit 12 is configured so that it may generate RF energy for use for treatment through electrodes and is configured to change the frequency, voltage, and the like of the RF energy as needed.

The temperature sensor unit 13 is configured to measure the output value of the RF energy, and is provided in an electrical path between the electrodes from the frequency unit 12 so that it may measure the current, voltage, and power applied to the electrodes.

The handpiece 10 may be provided with the temperature sensor unit 13 to calculate the impedance value of tissue, and the control unit 30 is configured to receive the sensed value from the temperature sensor unit 13 and control the frequency unit 12 and the like.

The temperature sensor unit 13 may stop radiofrequency output from the corresponding applicator 11 when the critical temperature initially set in the control unit 30 is detected.

The control unit 30 may determine the state of the tissue or the power of RF energy in response to the stored data according to the set user information.

The control unit 30 may be configured to determine the state of tissue through the temperature sensor unit 13, provided in the handpiece 10, during RF energy application, control the applied RF energy or block the RF energy, and control the frequency unit and other components when operating in various modes depending on a user's input.

Meanwhile, the handpiece 10 may further include a single ultrasonic vibration unit 40 capable of generating ultrasonic waves and vibrations, and the ultrasonic vibration unit 40 may adjust the number of repetitions for contraction and relaxation of muscles or may be operated alternately with the frequency unit 12.

Although not shown, the vibration unit may be mounted on one side of the handpiece 10, and may provide more effective radiofrequency treatment by providing a vibration massage function at the same time as generating deep heat in a body part through radiofrequency waves by the internal vibrator.

The vibration unit may be composed of a vibrating element that generates a vibration signal, a motor that supplies power to a vibrator according to the vibration signal, and a vibrator that generates vibration by the motor. When the applicator 11 is brought into close contact with a multi-joint part of the human body, vibration may be generated by the vibration unit to stimulate the human body part, thereby further enhancing the radiofrequency treatment effect on the sensory nerves of the human body part.

FIG. 3 shows the cycle of the operating frequency and duration of the handpiece 10 according to an embodiment of the present invention.

Referring to FIG. 3, according to an embodiment of the present invention, the handpiece may include six applicators 11, and among the plurality of applicators 11, a first group composed of Nos. 1 and 2, a second group composed of Nos. 3 and 4, and a third group composed of Nos. 5 and 6 may be operated in chronological order.

The control unit 30 may set the number of repetitions of the cycle according to the duration, and control the applicators 11 to increase the temperature of the body part stepwise while alternately operating each applicator 11 used in the cycle.

The control unit 30 may determine at least one applicator 11 set to be used for the body part, and operate to set the operating frequency and duration of each applicator 11 as a cycle according to the operating time.

Each cycle may be configured within the entire operating range. According to an embodiment of the present invention, the first cycle may be operated for 180 seconds with all groups operating for 60 seconds, and the second cycle may be operated for 90 seconds with all groups operating for 30 seconds. Similarly, the third cycle and the fourth cycle may operate for different operating times.

The total time may be set to 15 minutes, and the first to fourth cycles may be repeatedly reproduced or a specific cycle may be set to be repeated several times depending on a body part. Although the operating frequency is set to be the same for each cycle, the size thereof may be set to be variable and is not limited to the embodiment of the present invention.

Hereinafter, a control method for the above-described treatment apparatus using the radiofrequency handpiece 10 will be described.

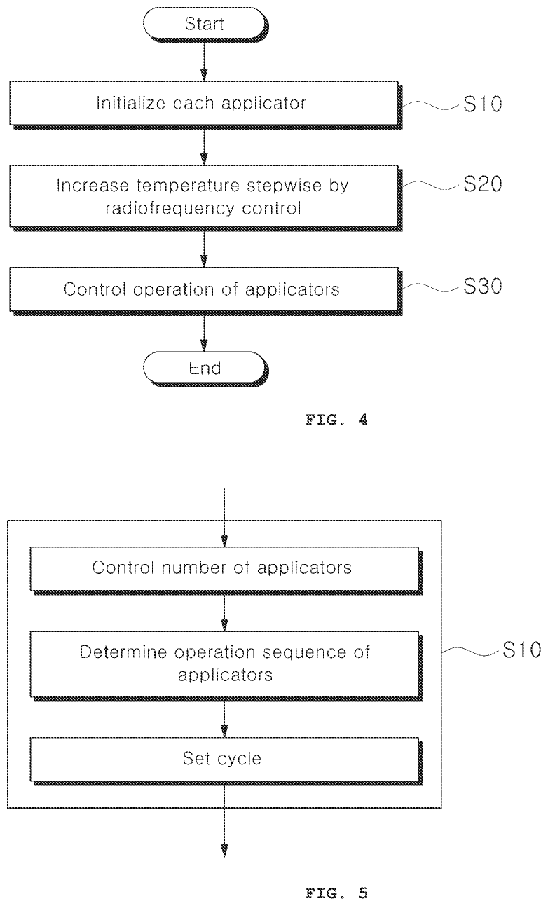

FIG. 4 is a flow chart showing the control method for the treatment apparatus using the radiofrequency handpiece 10 according to the present invention.

Referring to FIG. 4, the present invention may include steps of: initializing each applicator; increasing the temperature stepwise by radiofrequency control; and controlling the operation of the applicator.

Step (S10) of initializing each applicator is a process in which the control unit 30 receives, through the input unit 20, information on a user who is to be treated with a radiofrequency, and sets the treatment time and radiofrequency output of each applicator provided in the handpiece 10 depending on the user information.

The initialization process may include steps of: determining the number of the applicators using at least one of the user's age, gender, height, weight, treatment area, or body mass index (BMI) included in the user information; determining the operation sequence of each applicator depending on the number of the applicators; and setting the operating frequency and duration of each applicator as one cycle depending on the operation sequence.

As described above, the user information may include at least one of age, gender, height, weight, treatment area, or body mass index (BMI). Depending on the user information, the frequency of the radiofrequency may be initialized to operate stepwise during a preset treatment time.

Step (S20) of increasing the temperature stepwise by radiofrequency control is a process of generating a radiofrequency during the treatment time, detecting the ambient temperature, and increasing the temperature stepwise while controlling the radiofrequency depending on a preset time.

Step (S30) of controlling the operation of the applicators is a process of controlling the operation of the applicators included in the handpiece by receiving feedback from the user on the temperature depending on the output of the radiofrequency.

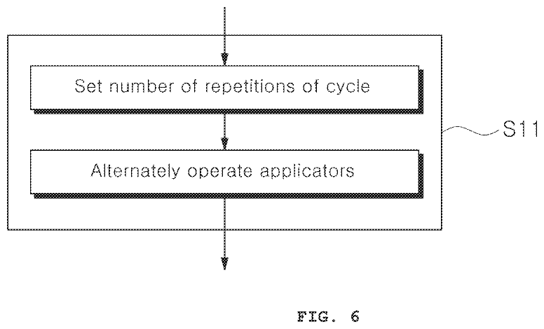

FIG. 5 shows a flow chart for initializing the treatment time and radiofrequency output of each applicator according to an embodiment of the present invention, and FIG. 6 shows a flow chart for increasing the temperature of a treatment area stepwise depending on an embodiment of the present invention.

Referring to FIG. 5, the initialization step may include steps of: allowing the control unit 30 to set the number of repetitions of the cycle depending on the duration; and alternately operating each applicator used in the cycle. According to the operation of the applicators, the temperature of the treatment area may be increased stepwise.

Referring to FIG. 6, according to the present invention, it is possible to set the number of repetitions of the cycle as described above with respect to the apparatus. That is, it is possible to set the number of repetitions of any specific cycle of the first to fourth cycles in the total operation time, which is usually described as 15 minutes. After the number of repetitions is set, it is possible to control the operation sequence of the first to third groups so that the operation of each applicator may occur alternately.

The present invention also provides a computer-readable recording medium storing a program for implementing the above-described control method as a program.

Although the present invention has been described above in detail through representative embodiments, those skilled in the art to which the present invention belongs will understand that various modifications to the above-described embodiments are possible without departing from the scope of the present invention. Therefore, the scope of the present invention should not be construed as being limited to the described embodiments, and should be defined not only by the appended claims, but also by all changed or modified forms derived from the claims and equivalents thereto.

1. A treatment apparatus using a radiofrequency handpiece, the apparatus comprising:
an input unit configured to receive information on a user who is to be treated with a radiofrequency;
a handpiece configured to generate the radiofrequency depending on the user information, detect an ambient temperature, and increase the temperature stepwise while controlling the radiofrequency depending on a preset time, the handpiece comprising a plurality of applicators having different radiofrequency output modes depending on a body part of the user; and
a control unit configured to control output of the radiofrequency, which is generated by the handpiece, depending on the temperature, and to control an operating mode of the handpiece through an impedance value of a treatment area, which is fed back from the user.

2. The treatment apparatus of claim 1, wherein the user information includes at least one of a gender, a height, a weight, a treatment area, or a body mass index (BMI) of the user, and depending on the user information, a frequency of the radiofrequency is initialized to operate stepwise during a preset treatment time.

3. The treatment apparatus of claim 1, wherein the plurality of applicators have at least one of a radiation pad, an electrode pad, a carbon sheet, or an air pad so as to operate in a monopolar radiofrequency manner and are attached in varying numbers depending on a body part of the user, and the handpiece further comprises:
a frequency unit configured to output a frequency set for each section of the treatment time; and
a temperature sensor unit configured to measure a temperature of the body part that increases stepwise depending on an output of the frequency unit.

4. The treatment apparatus of claim 3, wherein the handpiece further comprises a single ultrasonic vibration unit capable of generating ultrasonic waves and vibrations, and the ultrasonic vibration unit adjusts a number of repetitions for contraction and relaxation of muscles or is operated alternately with the frequency unit.

5. The treatment apparatus of claim 3, wherein the radiation pad and the electrode pad in the applicator further comprise a urethane sheet that comes into contact with skin, and the radiation pad has a positively charged carbon sheet attached to a lower surface of the urethane sheet, and a negatively charged carbon sheet attached to a lower surface of the electrode pad.

6. The treatment apparatus of claim 3, wherein the temperature sensor unit stops radiofrequency output from a corresponding applicator when a critical temperature initially set in the control unit is detected.

7. The treatment apparatus of claim 1, wherein the control unit determines at least one applicator set to be used for the body part, and sets an operating frequency and duration of each applicator as a cycle, depending on the operating time.

8. The treatment apparatus of claim 7, wherein the control unit sets a number of repetitions of the cycle according to the duration, and controls the applicators to increase the temperature of the body part stepwise while alternately operating each applicator used in the cycle.

9. A method for controlling the treatment apparatus of claim 1, the method comprising steps of:
allowing the control unit to receive, through the input unit, the information on the user who is to be treated with the radiofrequency, and to initialize the treatment time and radiofrequency output of each applicator, provided in the handpiece, depending on the user information;
generating a radiofrequency during the treatment time, detecting an ambient temperature, and increasing the temperature stepwise while controlling the radiofrequency depending on a preset time; and
controlling operation of the applicators comprised in the handpiece by receiving feedback from the user on the temperature depending on the output of the radiofrequency.

10. The method of claim 9, wherein the step of initializing the treatment time and radiofrequency output comprises steps of:
determining number of the applicators using at least one of a gender, a height, a weight, a treatment area, or body mass index (BMI) of the user included in the user information;
determining an operation sequence of each applicator depending on the number of the applicators; and
setting an operating frequency and duration of each applicator as one cycle depending on the operation sequence.

11. The method of claim 9, comprising steps of:
allowing the control unit to set a number of repetitions of a cycle depending on a duration; and
alternately operating each applicator used in the cycle, wherein the temperature of the treatment area is increased stepwise according to the operation of the applicators.

12. A non-transitory computer-readable recording medium storing a program for implementing the method of claim 9 as a program.
